(19) United States
(12) Patent Application Publication
(10) Pub. No.: US 2026/0325459 A1
(43) Pub. Date: Oct. 22, 2026

(54) POLYMER COATED ALKALINE EARTH PHOSPHATE NANOPARTICLE-BASED DELIVERY OF OLIGONUCLEOTIDES

(71) Applicant: Rutgers, The State University of New Jersey, New Brunswick, NJ (US)

(72) Inventors: Ki-Bum Lee, Monmouth Junction, NJ (US); Brandon Conklin, Fords, NJ (US)

(73) Assignee: Rutgers, The State University of New Jersey, New Brunswick, NJ (US)

(21) Appl. No.: 19/640,086

(22) Filed: Apr. 6, 2026

Related U.S. Application Data
(60) Provisional application No. 63/787,378, filed on Apr. 11, 2025.

(51) Int. Cl. A61K 9/51; A61K 31/7088; C12N 15/113; C12N 15/88

(52) U.S. Cl. CPC A61K 9/5153; A61K 9/5115; A61K 9/5192; A61K 31/7088; C12N 15/113; C12N 15/88; C12N 2310/127

(57) ABSTRACT

The present disclosure provides a biodegradable nanoparticle platform that encapsulates an oligonucleotide, such as a DNAzyme to suppress irf5 expression, which promotes macrophage polarization to an anti-inflammatory state. Herein, the ability to transfect classically activated macrophages, directly suppress IRF5 expression and downstream cytokines, and cause metabolic changes that are indicative of an alternatively activated phenotype is demonstrated. The nanoparticles disclosed herein offer a therapeutic approach to modulate inflammatory pathways in pro-inflammatory macrophages using a biodegradable and non-immunogenic platform.

### CROSS-REFERENCE TO RELATED APPLICATIONS

This application claims priority under 35 U.S.C. § 119 (e) to U.S. Provisional Patent Application No. 63/787,378, filed Apr. 11, 2025. The foregoing application is incorporated by reference herein in its entirety.

### STATEMENT REGARDING FEDERALLY SPONSORED RESEARCH OR DEVELOPMENT

This invention was made with government support under grant number NS130836 awarded by the National Institutes of Health and grant number HT9425-23-1-0365 awarded by the Defense Health Agency, Medical Research and Development Branch. The government has certain rights in the invention.

### REFERENCE TO AN ELECTRONIC SEQUENCE LISTING

This application incorporates by reference the Sequence Listing submitted in Computer Readable Form as file Seq_listing_07043901951.xml, created on Mar. 25, 2026 and containing 5,527 bytes.

### TECHNICAL FIELD

The present disclosure relates to biodegradable nanoparticle delivery systems for the targeted delivery of DNAzymes or other oligonucleotides to a cell to suppress mRNA expression.

### BACKGROUND

Inflammation is a complex biological response that depends on intricate communication networks between immune cells and the surrounding tissue microenvironment. While macrophages are key mediators of innate immune responses, delivering therapeutic oligonucleotides (nucleic acids) to these cells presents significant challenges that have yet to be overcome. Under typical conditions, unprotected nucleic acids are rapidly degraded by nucleases both inside and outside the cell, limiting their therapeutic efficacy.

DNA enzymes (DNAzyme), particularly the 10-23 DNAzyme, have shown promise in selectively targeting and degrading mRNA sequences. However, achieving efficient DNAzyme delivery and activation remains challenging due to the poor cellular uptake, instability in biological fluids, and dependence on intracellular divalent metal cofactors. While recent modifications have aimed to improve DNAzyme longevity and activity, many still rely on transfection reagents and intracellular concentration of divalent metal cofactors, resulting in poor kinetics and translatability.

### SUMMARY

Herein, a synergistic nanoparticle-based platform composed of alkaline earth phosphate nanoparticles coated with an ionizable polymer has been developed to address these limitations. This platform enhances DNAzyme stability, cellular uptake, and facilitates catalytic activation by providing a controlled release of divalent alkaline earth ions upon degradation.

In one aspect, the present disclosure provides a nanoparticle comprising: (a) a core comprising alkaline earth phosphate encapsulated oligonucleotides, and (b) an ionizable polymer enclosing the core, wherein the ionizable polymer is a biocompatible anionic polymer that ionizes at a pH between about 4 and about 6.5.

In another aspect, the disclosure provides a method of making the nanoparticle of any of the embodiments described herein, comprising:
(a) mixing two microemulsions, wherein the first microemulsion includes alkaline earth chloride and a DNAzyme, and wherein the second microemulsion includes disodium phosphate, and
(b) stirring the mixture of step (a) until alkaline earth phosphate particles encapsulating the DNAzyme are formed, (c) incubating a solution of an insoluble polymer and a base to form a solution of activated polymer, and (d) adding the alkaline earth phosphate particle formed in step (b) to the activated polymer solution in step (c) to form the polymer-coated nanoparticles.

In another aspect, the present disclosure provides a method of downregulating macrophage activation in a subject in need thereof, the method comprising administering to the subject an effective amount of the nanoparticle of any of the embodiments described herein.

In another aspect, the disclosure provides a method of transfecting a cell, the method comprising contacting the cell with an effective amount of the nanoparticle of any of the embodiments described herein. In some embodiments, the cell is a macrophage.

In another aspect, the present disclosure provides a 10-23 DNAzyme comprising a nucleotide sequence with at least 95% sequence identity to a nucleotide sequence selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca |

### BRIEF DESCRIPTION OF THE DRAWINGS

FIG. 1 shows a bar graph illustrating Time Dependent Influence of PCaP Treatment. THP-1 derived macrophages were activated with LPS and IFN-γ and treated with PCaP NP for 4-24 hours (n=3). Mean #SEM, one-way ANOVA, and turkey test. ****P<0.0001; n.s. not significant.

FIG. 2 shows a Western blot and quantified relative band intensity of unactivated, classically activated, and PCaP NP-treated macrophages for IRF5 and IL-1β (n=3). Mean±SEM one-way ANOVA, and turkey test.***P<0.001; *P<0.05

FIG. 3A shows Pearson correlation coefficient (PCC) demonstrating correlation between the dye labeled DNAzyme and endosome at various time points (n=3). FIG. 3B shows RT-qPCR demonstrating a decrease in nlrp3 when PCaP treated classically activated macrophages to highlight early endosomal escape (n=4). Mean±SEM, t-test, one-way ANOVA, and turkey test. ***P<0.001; **P<0.01; *P<0.05.

FIG. 4A shows a schematic demonstrating the relationship between IRF5 and IRF7. FIG. 4B shows RT-qPCR demonstrating a decrease irf7 expression in classically activated macrophages when treated with PCaP NP (n=3). Mean±SEM, one-way ANOVA and turkey test. *P<0.05; **P<0.01.

FIG. 5 shows Effects of PCaP-mediated IRF5 downregulation on Interferons. RT-qPCR demonstrating a decrease in ifn-β expression in classically activated macrophages when treated with PCaP NP (n=4). Mean±SEM, one-way ANOVA and turkey test. ***P<0.001; n.s. not significant.

FIG. 6 shows a representative qNMR spectra used to identify key metabolites and schematic illustration of how these spectra are used to make biological interpretations about cellular behavior following treatment.

FIG. 7 shows a PLS-DA analysis and 95% confidence intervals demonstrating PCaP NP-treated classically activated macrophages are metabolically distinct from M1-like and unactivated macrophages. Important metabolites were identified via the partial least squares discriminant analysis (PLS-DA) loading scores (n=3).

FIG. 8A is a bar graph showing the ratio of ATP:AMP demonstrating changes in bioenergetics following PCaP NP treatment (n=3). FIG. 8B is a bar graph showing the changes in glucose as quantified by qNMR. Mean±SEM one-way ANOVA and turkey test. **P<0.01; n.s. not significant.

FIG. 9 is a bar graph showing the quantification of images for the J-aggregate: JC-1 Monomer ratio (n=3). Mean±SEM, one-way ANOVA and turkey test. ***P<0.001; **P<0.01; *P<0.05

FIG. 10 is a bar graph showing the RT-qPCR of HIF-1α to further demonstrate mitochondrial health in classically activated (n=4) and treated (n=3) macrophages. Mean±SEM, t-test. **P<0.01.

FIG. 11 shows a representative mass spectrometry spectra demonstrating differences in lipidomic profiles in M0, M1-like, and M1-like treated macrophages with PCaP NP. The data was subject to median normalization, log transformation, and auto-scaled to create a normal distribution.

FIG. 12 is a plot showing the Partial Least Squares Discriminant Analysis (PLS-DA) scores of M1-like and M1-like treated macrophages (n=3).

FIG. 13 shows the pathway enrichment analysis of M1-like and M1-like treated macrophages (n=3).

FIG. 14A is a bar graph showing the RT-qPCR results of CPT1 expression between classically activated and treated macrophages validating the carnitine shuttle enrichment. FIG. 14B is a bar graph showing the RT-qPCR results of CPT2 expression between classically activated and treated macrophages validating the carnitine shuttle enrichment. Mean±SEM, t-test. ***P<0.001; ****P<0.0001.

FIG. 15A is a volcano plot showing genes regulated in classically activated versus unactivated macrophages. FIG. 15B is a volcano plot showing genes regulated in classically activated versus PCaP NP treated classically activated macrophages (n=5).

FIG. 16 is a PCA analysis demonstrating M0, M1-like, and M1-like treated macrophages possess distinct transcriptomic signatures (n=5).

FIG. 17A is a bar graph showing genes that were differentially expressed in M1-like treated macrophages compared to M1-like macrophages were entered into pathway analysis for biological processes. FIG. 17B is a bar graph showing genes that were differentially expressed in M1-like treated macrophages compared to M1-like macrophages were entered into pathway analysis for molecular processes. FIG. 17C is a bar graph showing genes that were differentially expressed in M1-like treated macrophages compared to M1-like macrophages were entered into pathway analysis for cellular processes (n=5).

FIG. 18A shows a pie chart demonstrating the percent of proinflammatory and anti-inflammatory genes regulated in classically activated macrophages. FIG. 18B shows a pie chart demonstrating the percent of proinflammatory and anti-inflammatory genes regulated in PCaP NP treated macrophages.

FIG. 19 is a Venn diagram showing the unique and overlapped genes in the top 5 biological processes.

### DETAILED DESCRIPTION

Herein, the present disclosure provides a biodegradable nanoparticle platform that encapsulates an oligonucleotide, such as a DNAzyme to suppress irf5 expression, which promotes macrophage polarization to an anti-inflammatory state. Herein, the ability to transfect classically activated macrophages, directly suppress IRF5 expression and downstream cytokines, and cause metabolic changes that are indicative of an alternatively activated phenotype is demonstrated. The nanoparticle disclosed herein offer a therapeutic approach to modulate inflammatory pathways in pro-inflammatory macrophages using a biodegradable and non-immunogenic platform.

The articles “a” and “an” as used herein refers to “one or more” or “at least one,” unless otherwise indicated. That is, reference to any element or component of an embodiment by the indefinite article “a” or “an” does not exclude the possibility that more than one element or component is present.

The term “about” as used herein refers to the referenced numeric indication plus or minus 10% of that referenced numeric indication.

A “therapeutically effective amount” of the nanoparticle described herein may vary according to factors such as the disease state, age, sex, and weight of the individual, and the ability of the nanoparticle to elicit a desired response in the individual. A therapeutically effective amount is also one in which any toxic or detrimental effects of the nanoparticle are outweighed by the therapeutically beneficial effects. The term “therapeutically effective amount” refers to an amount of the nanoparticle or composition comprising the same, which is effective to treat a disease or disorder in a subject (e.g., a patient).

“Treat,” “Treatment,” “treating,” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in subjects. “Treatment” or “treating” does not necessarily indicate complete eradication or cure of the disease or condition, or associated symptoms thereof.

The “subject” receiving this treatment is intended to include human and non-human animals. Non-human animals include all vertebrates, e.g. mammals and non-mammals, such as non-human primates, sheep, dogs, cats, cows, horses, chickens, amphibians, and reptiles, although mammals are preferred, such as non-human primates, sheep, dogs, cats, cows and horses.

In one aspect, the present disclosure provides a nanoparticle comprising: (a) a core comprising alkaline earth phosphate encapsulated oligonucleotides, and (b) an ionizable polymer enclosing the core, wherein the ionizable polymer is a biocompatible anionic polymer that ionizes at a pH between about 4 and about 6.5.

The nanoparticles described herein comprise a core comprising an alkaline earth phosphate. The nanoparticle core protects the oligonucleotide cargo from degradation by extracellular and intracellular nucleases. Further, particularly for DNAzymes, not only does the alkaline earth phosphate core protect the DNAzyme cargo but also provides a continuous source of divalent alkaline earth ions (e.g. Mg²⁺ or Ca²⁺) that enhance DNAzyme activity. In some embodiments, the alkaline earth phosphate is magnesium phosphate. In some embodiments, the alkaline earth phosphate is calcium phosphate.

The alkaline earth core can be doped with one or more additional divalent ions to improve biological and material properties. In some embodiments, doping with one or more divalent ions provides anti-bacterial and anti-viral properties. In some embodiments, doping with one or more divalent ions increases the catalytic activity of the DNAzyme and accelerates wound healing. In some embodiments, the alkaline earth phosphate core is doped with one or more additional divalent metal ion(s). In some embodiments, the divalent metal ion is selected from Zn²⁺ and Mn²⁺.

The core encapsulates an oligonucleotide, which provides a therapeutic effect upon transfection into a cell, by modulating gene expression, splicing, and/or other intracellular processes. In some embodiments, the oligonucleotide is selected from the group consisting of miRNAs, siRNAs, plasmids, shRNA, peptide nucleic acids (PNA), aptamers and DNAzymes.

In some embodiments, the oligonucleotide is a DNAzyme. A DNAzyme is a synthetic DNA molecule with catalytic activity that can bind specific RNA or DNA sequences and provides a therapeutic effect. In some embodiments, the DNAzyme is a 10-23 DNAzyme that suppresses an mRNA encoding the protein Interferon Regulatory Factor 5 (IRF5).

In some embodiments, the 10-23 DNAzyme includes a nucleotide sequence with at least 95% sequence identity to a nucleotide sequence selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca. |

The nanoparticles are further coated with an ionizable polymer, which is a biocompatible anionic polymer that ionizes at a pH between about 4 and about 6.5. The ionizable polymer is electrostatically attached to the nanoparticle surface and helps promote electrostatic interactions with the cell membrane, enhancing cellular uptake and facilitating endosomal escape. In some embodiments, the anionic polymer ionizes at a pH of about 4.0, about 4.5, about 5.0, about 5.5, about 6.0, about 6.5, or any range or value between any two of the aforementioned pH values.

In some embodiments, the ionizable polymer is a poly(amino acid)-based polymer or co-polymer, a biopolymer, a polylactide-polyethylene glycol (PLA-PEG), or a zwitterionic polymer or co-polymer, wherein the poly(amino acid)-based polymer or co-polymer is selected from the group consisting of poly(β-amino ester) (PBAE), poly(aspartic acid-co-lysine), poly(glutamic acid-co-lysine), and poly(aspartic acid-co-histidine); wherein the biopolymer is selected from the group consisting of gelatin type B, collagen, casein, and peptidoglycan; and wherein the zwitterionic polymer or co-polymer is selected from the group consisting of poly(carboxybetaine methacrylate) and poly(acrylic acid-co-dimethylaminoethyl methacrylate). In some embodiments, the ionizable polymer is a poly(β-amino ester) (PBAE).

In some embodiments, the nanoparticles have an average size of about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, and any range between any two of the aforementioned values, as characterized by transmission electron microscopy.

In another aspect, the disclosure provides a method of making the nanoparticle of any of the embodiments described herein, comprising:
(a) mixing two microemulsions, wherein the first microemulsion includes an alkaline earth halide and a DNAzyme, and wherein the second microemulsion includes disodium phosphate, and
(b) stirring the mixture of step (a) until alkaline earth phosphate particles encapsulating the DNAzyme are formed, (c) incubating a solution of an insoluble polymer and a base to form a solution of activated polymer, and (d) adding the alkaline earth phosphate particle formed in step (b) to the activated polymer solution in step (c) to form the polymer-coated nanoparticles.

In some embodiments, the alkaline earth phosphate encapsulated DNAzyme particles formed in step (b) are washed with a solvent comprising ethanol. In some embodiments, the base in step (b) is sodium acetate.

In some embodiments, the alkaline earth halide comprises a cation selected from magnesium (Mg²⁺) and calcium (Ca²⁺). In some embodiments, the alkaline earth halide comprises a halide selected from fluoride (F⁻), chloride (Cl⁻), bromide (Br) and iodide (I⁻).

In some embodiments, the insoluble polymer is a poly(amino acid)-based polymer or co-polymer, a biopolymer, a polylactide-polyethylene glycol (PLA-PEG), or a zwitterionic polymer or co-polymer, wherein the poly(amino acid)-based polymer or co-polymer is selected from the group consisting of poly(β-amino ester) (PBAE), poly(aspartic acid-co-lysine), poly(glutamic acid-co-lysine), and poly(aspartic acid-co-histidine); wherein the biopolymer is selected from the group consisting of gelatin type B, collagen, casein, and peptidoglycan; and wherein the zwitterionic polymer or co-polymer is selected from the group consisting of poly(carboxybetaine methacrylate) and poly(acrylic acid-co-dimethylaminoethyl methacrylate). In some embodiments, the insoluble polymer is a poly(β-amino ester) (PBAE).

In another aspect, the present disclosure provides a method of downregulating macrophage activation in a subject in need thereof, the method comprising administering to the subject an effective amount of the nanoparticle of any of the embodiments described herein.

In some embodiments, the nanoparticle is administered systemically. In some embodiments, the nanoparticle is administered locally

In some embodiments, the nanoparticle is administered to treat neuropathic pain, an autoimmune disease, allograft rejection or Graft-versus-Host Disease (GVHD).

In some embodiments, the autoimmune disease is selected from the group consisting of Acute Disseminated Encephalomyelitis, Agammaglobulinemia, Allergic Disease, Ankylosing spondylitis, Anti-GBM/Anti-TBM nephritis, Anti-phospholipid syndrome, Autoimmune aplastic anemia, Autoimmune hepatitis, Autoimmune inner ear disease, Autoimmune myocarditis, Autoimmune pancreatitis, Autoimmune retinopathy, Autoimmune thrombocytopenia purpura, Behcet's Disease, Bullous pemphigoid, Castleman's Disease, Celiac Disease, Churg-Strauss syndrome, Crohn's Disease, Cogan's syndrome, Dry eye syndrome, Essential mixed cryoglobulinemia, Dermatomyositis, Devic's Disease, Encephalitis, Eosinophlic esophagitis, Eosinophilic fasciitis, Erythema nodosum, Giant cell arteritis, Glomerulonephritis, Goodpasture's syndrome, Granulomatosis with Polyangiitis (Wegener's), Graves' Disease, Guillain-Barre syndrome, Hashimoto's thyroiditis, Hemolytic anemia, Henoch-Schonlein purpura, IgA nephropathy, Inclusion body myositis, Type I diabetes, Interstitial cystitis, Kawasaki's Disease, Leukocytoclastic vasculitis, Lichen planus, Systemic lupus erythematosus (SLE), Microscopic polyangitis, Multiple sclerosis, Myasthenia gravis, myositis, Optic neuritis, Pemphigus, POEMS syndrome, Polyarteritis nodosa, Primary biliary cirrhosis, Psoriasis, Psoriatic arthritis, Pyoderma gangrenosum, Relapsing polychondritis, Rheumatoid arthritis, Sarcoidosis, Scleroderma, Sjogren's syndrome, Takayasu's arteritis, Transverse myelitis, Ulcerative colitis, Uveitis, and Vitiligo.

Treatment of neuropathic pain, an autoimmune disease, allograft rejection or Graft-versus-Host Disease (GVHD) herein is intended to also include therapeutic administration of a nanoparticle of the present disclosure, or a pharmaceutical salt thereof, or a pharmaceutical composition of either to a subject which may be in need of preventative treatment, such as, for example, for pain, inflammation and the like. Treatment also encompasses prophylactic administration of a nanoparticle of the present disclosure, or a pharmaceutical salt thereof, or a pharmaceutical composition of to a subject. Generally, the subject is initially diagnosed by a licensed physician and/or authorized medical practitioner, and a regimen for prophylactic and/or therapeutic treatment via administration of the nanoparticle(s) or compositions of the present disclosure is suggested, recommended or prescribed.

The amount of nanoparticle(s) which is/are administered and the dosage regimen for treating neurodegenerative diseases with the nanoparticle(s) and/or compositions of the present disclosure depends on a variety of factors, including the age, weight, sex and medical condition of the subject, the type of disease, the severity of the disease, the route and frequency of administration, and the particular nanoparticle employed. Thus, the dosage regimen may vary widely but can be determined routinely using standard methods. A daily dose of about 0.01 to 500 mg/kg, advantageously between about 0.01 and about 50 mg/kg, more advantageously about 0.01 and about 30 mg/kg, and even more advantageously between about 0.1 and about 10 mg/kg body weight may be appropriate and should be useful for all methods of use disclosed herein. The daily dose can be administered in one to four doses per day.

While it may be possible to administer the nanoparticle(s) of the present disclosure alone, in the methods described, the nanoparticle administered normally will be present as an active ingredient in a pharmaceutical composition. Thus, in some embodiments, the present disclosure provides a pharmaceutical composition comprising any of the aforementioned nanoparticle(s) in combination with a pharmaceutically acceptable excipient, which includes diluents, carriers, adjuvants and the like (collectively referred to herein as “excipient” materials) as described herein, and, if desired, other active ingredients. A pharmaceutical composition of the present disclosure may comprise an “effective amount” of a nanoparticle or an “effective dosage amount” of a nanoparticle. An “effective dosage amount” of a nanoparticle includes an amount less than, equal to or greater than an effective amount of the nanoparticle. For example, a pharmaceutical composition in which two or more unit dosages, such as in tablets, capsules and the like, are required to administer an effective amount of the nanoparticle, or alternatively, a multi-dose pharmaceutical composition, such as powders, liquids and the like, in which an effective amount of the nanoparticle is administered by administering a portion of the composition.

The nanoparticle(s) of the present disclosure may be administered by any suitable route, preferably in the form of a pharmaceutical composition adapted to such a route, and in a dose effective for the treatment intended. The nanoparticles and compositions of the present disclosure may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, advantageously from about 1 to 500 mg, and typically from about 5 to 150 mg. A suitable daily dose for a human or other mammal may vary widely depending on the condition of the patient and other factors, but, once again, can be determined using routine methods and practices.

For therapeutic purposes, the active nanoparticle(s) are ordinarily combined with one or more adjuvants or other “excipients” appropriate to the indicated route of administration. If orally administered on a per dose basis, the nanoparticles may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, to form the final formulation. For example, the active nanoparticle(s) and excipient(s) may be tableted or encapsulated by known and accepted methods for convenient administration. Examples of suitable formulations include, without limitation, pills, tablets, soft and hard-shell gel capsules, troches, orally-dissolvable forms and delayed or controlled-release formulations thereof. Particularly, capsule or tablet formulations may contain one or more controlled-release agents, such as hydroxypropylmethyl cellulose, as a dispersion with the active nanoparticle(s).

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The nanoparticles may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (ie. propylene glycol) or micellar solubilization (ie. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

The active ingredient may also be administered by injection as a composition with suitable carriers including saline, dextrose, or water. The daily parenteral dosage regimen will be from about 0.1 to about 30 mg/kg of total body weight, and preferably from about 0.1 to about 10 mg/kg.

For pulmonary administration, the pharmaceutical composition may be administered in the form of an aerosol or with an inhaler including dry powder aerosol.

The pharmaceutical compositions may be subjected to conventional pharmaceutical operations such as sterilization and/or may contain conventional adjuvants, such as preservatives, stabilizers, wetting agents, emulsifiers, buffers etc.

Tablets and pills can additionally be prepared with enteric coatings. Such compositions may also comprise adjuvants, such as wetting, sweetening, flavoring, and perfuming agents. Accordingly, in some embodiments, the present disclosure provides a method of manufacturing a medicament, the method comprising combining an amount of any of the aforementioned nanoparticle(s) with a pharmaceutically acceptable carrier to manufacture the medicament.

The nanoparticle or pharmaceutical composition may be adapted for administration by any appropriate topical route, including buccal, sublingual or transdermal. Such compositions may be prepared by any method known in the art of pharmacy, for example by admixing the active ingredient with the carrier(s) or excipient(s) under sterile conditions.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3 (6): 3 18 (1986).

Pharmaceutical compositions adapted for topical administration may be formulated as ointments, creams, suspensions, lotions, powders, solutions, pastes, gels, sprays, aerosols or oils. For treatment of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

In another aspect, the disclosure provides a method of transfecting a cell, the method comprising contacting the cell with an effective amount of the nanoparticle of any of the embodiments described herein. In some embodiments, the cell is a macrophage.

In another aspect, the present disclosure provides a 10-23 DNAzyme comprising a nucleotide sequence with at least 95% sequence identity to a nucleotide sequence selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca |

In some embodiments, the nucleotide sequence is selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca |

### EXAMPLES

### Materials and Methods

### Gene Expression Analysis

The expression levels of target genes were quantified by quantitative reverse transcription polymerase chain reaction RT-qPCR of mRNA extracted from cell culture. The total RNA was extracted using TRIzol Reagent (Life Technologies) and 1 μg of the total RNA was used as a template for a reverse transcription reaction to generate the first-strand complementary cDNA using Superscript III First-strand Synthesis System (Life Technologies) according to the manufacturer's protocol. The first strand cDNA was then used for a qPCR reaction with gene-specific primers in the presence of Power SYBR Green PCR Master Mix (Applied Biosystems) on a StepOnePLus Real-Time PCR System (Applied Biosystems). The Ct values were normalized to GAPDH, which was selected as the endogenous control. The standard cycling conditions were applied to all the reactions conditions with a melting temperature of 60° C. All gene-specific primers were obtained from the PrimerBank database . . . .

### Transfection and Immunostaining Quantification

The proportion of green-positive or red-positive cells were calculated using an automated method run with python slightly modified from Guo, C. et. al., CellTag Indexing: genetic barcode-based sample multiplexing for single-cell genomics. Genome Biol 2019, 20 (1), 90. The background image was translated into binary images by thresholding the brightfield contrast. Specifically, the Otsu method was utilized to determine the threshold for detecting a contrast. The watershed method was used to segment the binary image into individual cells. The detected cells were then filtered to remove cell aggregates and debris. The intensity of the fluorescent signal of interest [red/green] was then quantified to determine the number of fluorescent positive cells and ratio of fluorescent cells to non-fluorescent cells in the processed image. The processes were run with the Python 3.11.1 and its libraries: scikit-image 0.19.2, numpy 1.24.0, matplotlib 3.6.2, seaborn 0.12.2, jupyter 5.2.

DNAzyme Kinetic Studies with Denaturing PAGE

For multiple turnover experiments stock solutions of 2 μM DNAzyme and 20 μM RNA substrate were created. The substrate and DNAzyme were combined and bound together at 90 C for 10 minutes and then slowly cooled at room temperature according to established protocols for RNA-ssDNA binding. DNAzyme and the target sequence were then cleaved in a time dependent manner following the addition of CaCl₂) such that the final concentration of CaCl₂) was 1 mM. The cleavage event was quenched at each time point by diluting the sample 10 with a solution of 25 mM EDTA in 99% formamide. Sample secondary structures were denatured by heating at 95 Celsius for 15 minutes followed by ice for separation of bound products. A 30% denaturing PAGE gel was prepared with 50% urea solution by weight (w/v) in 1×TBE buffer with 40% acrylamide/bis 1:29 solution. The gel was run at 5 V/cm². The gel was analyzed with a Thermo Fischer iBright UV illuminator and quantified with Thermo Fischer iBright analysis software [version 5.2.2]

### THP-1 Cell Culture, Macrophage Differentiation, and M1 Polarization

THP-1 monocytes were cultured in complete RPMI-1640 medium (Gibco, ThermoFisher Scientific) with 10% FBS, 1% penicillin-streptomycin and 0.1% β-mercaptoethanol. The THP-1 cells were differentiated into macrophage-like cells with 50 ng/ml phorbol 12-myristate 13-acetate (PMA) (TocrisBioscience) for 48 h. For polarization into M1-like macrophages, the cells were incubated with 100 ng mL-1 LPS and 50 ng/ml IFN-γ for 24 h after differentiation.

### X-Ray Photoelectron Spectroscopy (XPS)

Silicon substrates (1 cm×1 cm) cut from Prime-grade silicon wafers were used for X-ray photoemission spectroscopy (XPS) sample preparation. The substrates were thoroughly cleaned by sonicating them for 30 minutes in a 1% Triton X-100 solution, followed by sonicating in DI water for 20 minutes and ethanol for 20 minutes. They were then dried in a vacuum desiccator. PCaP solutions were drop-cast onto the cleaned silicon substrates with a minimum volume of 10 μL and dried overnight under vacuum at room temperature.

XPS measurements were performed on a Thermo K-alpha instrument using a monochromatized Al Kα photon source with a 400 μm x-ray spot and photon energy of 1486.7 eV. Survey spectra and core-level spectra were recorded in constant analyzer energy mode with pass energies of 200 eV and 50 eV, and energy step sizes of 1.00 eV and 0.100 eV, respectively. Core-level spectra were charge-corrected and referenced to the binding energy of the C—C bond at 284.8 eV. Data analysis, including element identification and peak fitting, was conducted using the Avantage Data System.

### Cell Metabolite Extraction

The extraction process began after polarizing the THP-1 cells. First, the cells were washed with 4 mL of ice-cold PBS to remove any unwanted cellular debris or dead cells before fixation. Once washed, the cells were fixed using 4 mL of ice-cold 1:1 methanol:water for 10 minutes. Using a cell scraper, these cells were thoroughly and mechanically unadhered from the dish within the fixative solution. After scraping, 3.5 mL of the resulting cell-solution was pipetted into relevantly labeled 15-mL conical tubes and kept on ice. Quickly after transfer, 10 uL of the cell solution was removed for cell-counting using an ADAM MC2 NanoEnTek. The cell samples were then lysed further using a probe ultrasonicator with the following experimental parameters: 30 second experiment time, 10 seconds on/2 seconds off, 30% amplitude. To prevent sample overheating, this sonication as performed over ice. After sonication, there was a methanolic-aqueous cell extract which needed further purification to separate cytosolic from lipid cellular metabolites that remained. To each methanolic-aqueous extract was added half the volume of chloroform (For 3.5 mL methanolic-aqueous extract in this case, 1.75 mL of chloroform was used). Immediately after addition, each solution was vigorously vortexed 5 times for 10 seconds each. The samples were stored in a −20° C. freezer for 20 minutes to allow the solvent layers to form. Afterwards, the samples were centrifuged for 10 minutes at 10,000 rpm to further separate the solvent layers. A second set of empty 15-mL conical tubes were subsequently labeled as methanolic-aqueous extracts for the corresponding experimental condition. Additionally, an equivalent number of glass vials were labeled as chloroform extracts for the corresponding experimental condition. For each centrifuged sample, exactly 3.2 mL of the top methanolic-aqueous layer was carefully removed and pipetted into its newly labeled and associated tube. Likewise, exactly 1.5 mL of the bottom chloroform layer was transferred in the same manner to the associated glass vials. Once transferred, all labeled samples were relocated to a −80° C. freezer for long-term storage.

### Preparation of Metabolite Extract for LC-MS

Chloroform was removed from the metabolite extracts using the rotary evaporator. The samples were stored as a powder at −80 C. The samples were then dissolved in methanol with 0.1% acetic acid, vortexed for several minutes, and stored at 10 C in preparation for data acquisition by LC-MS.

qNMR Cytosolic-Cell Metabolite Analysis

For analysis utilizing NMR, all protonated water and methanol must be removed from the sample as to not interfere with analysis. To do this, the samples were lyophilized. The lyophilizer was brought down to low vacuum with the associated cold-trap held at roughly-80° C. Once stabilized, the samples were swiftly removed from the −80° C. freezer, uncapped, and loaded into the vacuum chambers. The samples were introduced to the low vacuum and were left to lyophilize overnight. The next day, the tubes now containing a dry, white, powder were removed from the lyophilizer and capped to prevent the accumulation of moisture. For qNMR analysis, a sodium phosphate deuterium oxide (1 mM Na₂HPO₄/NaH₂PO₄, 7.4 pH, D₂O) buffer was prepared to prevent pH shifts induced by the sample. Maleic acid (Sigma-Aldrich TraceCERT®, lot #BCCC6481) was chosen as the internal standard used for quantification due to its water solubility, singular proton signal, and proton ppm shift. A 10 mg/mL maleic acid (MA) solution was prepared using the 1 mM Na₂HPO₄/NaH₂PO₄ D₂O buffer, and kept in the −20° C. freezer for long term storage. To prepare each sample for qNMR analysis, the lyophilized powder was dissolved in 590 μL of 1 mM Na₂HPO₄/NaH₂PO₄ D₂O buffer and transferred to a clean and dry 5 mm glass NMR tube. To this NMR tube was added 10 μL of MA solution directly, for a total of 600 μL per sample. The samples were analyzed on a Bruker Avance Neo 500 MHz with a RT-DR-BF/1H-5 mm-Z-SP iProbe with automatic tuning and 24 sample case auto sample changer. TopSpin 4.1.0 software was used for spectral acquisition. For the utilization of MA as internal standard for quantitative measurement, a 90° pulse with a relaxation delay of 44 seconds (MA T₁=6.3) was deployed to ensure full relaxation of nuclei from their excited state back down to ground. For qNMR analysis, the investigator must wait 7× the longest T₁ of the signals of interest (In this case, MA) before pulsing again for recording the next scan. For suitable signal-to-noise ratios, each experiment was ran for 128 scans before termination and processing. All processing was undergone in TopSpin 4.1.0 software, and all qNMR calculation was done by utilizing the following equation:

$${P}_{sample} = \frac{{S}_{sample}}{{S}_{std}} \times \frac{{N}_{std}}{{N}_{sample}} \times \frac{{m}_{std}}{{m}_{sample}} \times \frac{{M}_{sample}}{{M}_{std}} \times {P}_{std}$$

Where S=Integrated area of the peak, N=Number of protons (Number of hydrogen atoms in the functional group), m=Prepared mass, M=Molecular weight, and P=Purity. For the purposes of this disclosure, all cell metabolite was normalized quantification to cell counts instead of sample mass. In this way, the moles of each signal of interest was calculated before normalizing the value to the cell count (i.e., nanomoles/million cells) instead of relying on the weight of sample produced from lyophilization. All equations and calculations were embedded and conducted in Microsoft Excel.

### LC-MS Analysis of Metabolite Extracts

Samples were first run for quality control to ensure extract quality. Reverse phase liquid chromatography (RPLC) was performed on Acquity UPLC, BEHC18, 1.7 micron (2.1×50 mm) column Waters Corporation (Milford, MA). Column temperature was set at 40 C. The following mobile phase were used A: Water with 0.1% formic acid; B: acetonitrile with 0.1% formic acid. The UPLC H-Class high flow LC (Waters Instruments) was operated at a flow rate of 0.4 mL/min, and 8 μL of sample was injected with an autosampler after equilibration of the column. For the separation of compounds, the following gradient concentrations was used starting with 5-95% B for 3.5 minutes and later to 5% B from 3.5-5 min, for column equilibration. Prior to injection on the RPC column, a lock spray sample or reference sample or QC (Leucine-Enkephalin Dipeptide) run was performed. Total run time of the LC-MS run was set for 5 minutes.

Metabolite samples were then run. The mobile phase consisted of (A) 0.1% acetic acid and water and (B) 0.1% acetic acid and acetonitrile. The flow rate was 300 μL/min, and the injection volume was 10 using autosampler. A gradient with the following properties was used: 5=0-0.5 min 1% B; t=11 min, 99% B; 5=15.50 min, 99% B; t=15.60 min, 1% B. The total run time was 20 minutes. ESI-MS experiments were performed on high resolution mass spectrometer (HRMS) in a sensitivity mode using a quadrupole-TOF hybrid mass spectrometer (Xevo-G2-XS-Q-tof: Waters Corp., Manchester, UK) in positive ionization mode with m/z scan range 100-2000. The applied experimental parameters were capillary voltage, 3.18 kV; sampling cone voltage, 30 V; source offset 60° C.; source temperature, 125° C.; desolvation temperature 300° C.: Cone gas 25 L/hour and desolvation gas 600 L/hour. Data analysis was performed using MassLynx 4.1. The mass accuracies of the compounds were calculated by running lock spray standard.

### File Preparation for MetaboAnalyst 6.0

Raw data files were converted into mzML files using Proteo Wizard MS converter (version 3) and was processed using the MetaboAnalystR 4.0.0 library in RStudio. The data processing with this library involved using the centWave method for feature detection, optimizing parameters (i.e., ppm=22.5 ppm, minimum peak width=3.75 seconds, maximum peak width=45 s, signal/noise threshold=12.5, and mzdiff=0.0108, bw=2, minimum Fraction=0.8, minimum samples=1), peak profiling, peak annotation, and formatting the peak list. The outputted peak table was used as the input for online processing and enrichment analysis performed using MetaboAnalyst 6.0. The inputted data was filtered using an interquartile range of 40% prior to performing median normalization, log transformation, and auto scaling before performing analysis.

### Example 1

### Synthesis and of Polymer-Coated Calcium Phosphate Nanoparticles (PCaP NPs)

### Synthesis of Calcium Phosphate (CaP) Nanoparticles

Two separate microemulsions were formed using cyclohexane, Igepal CO-520, and water. For microemulsion 1, the aqueous phase consisted of 150 μL of CaCl₂) (500 mM) and 10 μL of IRF5 DNAzyme (1 μg*μL⁻¹ IDTE pH 8.0) that were incubated for 5 mins at room temperature. Following the incubation, the aqueous solution was added to 5 mL of a mixture of (3:7 vol/vol) Igepal CO-520: cyclohexane for 10 mins in a 50 mL flask while stirring at 350 rpm. Microemulsion 2 was formed in a separate 50 mL flask with an aqueous phase consisting of 150 μL of disodium phosphate (25 mM) added to 5 mL of a mixture of (3:7 vol/vol) Igepal CO-520: cyclohexane for 10 mins. To form the CaP nanoparticles, microemulsion 2, containing the disodium phosphate, was added to microemulsion 1 in 1 mL increments and allowed to stir at 350 rpm for 10 mins. To improve the stability of the particles, 20 μL of sodium citrate (200 mM) was added to the flask containing Cap and was allowed to stir for 30 mins. From here, 10 mL of ethanol was added to the citrate coated CaP particles, which were then washed via centrifugation for 20 mins at 10,000 g. Following the initial wash and immediately decanting the supernatant, the particles were dispersed in 10 mL of ethanol and washed twice more under the same conditions before being dispersed in 1 mL of water. The particles were centrifuged 5 mins at 13,300 g to remove excess ethanol and the particle pellet was immediately dispersed in 200 μL of sodium citrate (20 mM) producing a 5 mg*mL⁻¹ CaP solution.

### Polymer Coating of CaP Nanoparticle

The coating of CaP with PBAE was performed under sterile conditions to mitigate the chance of contamination when delivered to THP-1 macrophages. First, the PBAE was activated using sodium acetate. Specifically, 1 μL of PBAE (30 mg*mL⁻¹) solution in DMSO was added to 20 μL of pH 5.0 sodium acetate (20 mM) and left to incubate for 10 mins at room temperature to activate the polymer³. Next, 100 μg of CaP (20 μL) was added to the activated polymer solution and left at room temperature for 5 mins to create PCaP. A solution of 100 ng*μL⁻¹ PCaP was delivered to THP-1 macrophages for all experiments.

### Example 2

### Optimizing the IRF5 DNAzyme to Attenuate Inflammatory Responses in Classically Activated Macrophages

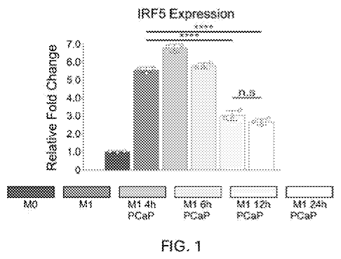

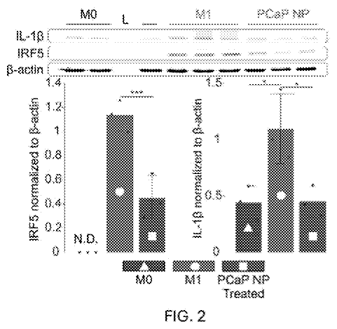

Many chronic diseases and injuries stem from poorly regulated inflammatory pathways that fail to resolve over time. As a result, pinpointing molecular factors that can help control these imbalanced responses is vital for the development of effective anti-inflammatory therapies. By better understanding the underlying mechanisms driving persistent inflammation, researchers can design targeted interventions to restore balance and promote tissue repair. Previously, the transcription factor IRF5 has been shown to promote classical activation of macrophages by mediating cytokine expression and glycolytic pathways. The function of IRF5 is specific to promoting inflammation as opposed to other candidates, such as NF-κB, that promote inflammation but are also vital to cellular function. Applicant's PCaP NP platform was designed to synergistically deliver and activate a DNAzyme that can recognize and cleave target mRNA for IRF5 following macrophage activation. However, several aspects of the DNAzyme's design needed to be optimized to ensure its efficient target cleavage and catalytic recycling. These factors included the specific cleavage site selected within the target mRNA and the length of the DNAzyme's binding arms, both of which influence its overall activity. Applicant began by dividing the irf5 sequence into 750 bp fragments and input these fragments into the open-source software MXfold2 to predict the secondary structure of the mRNA sequence. From these secondary structures, Applicant identified potential target sites in the mRNA that had guanine (G) and uracil (U) adjacent to one another in a location that was not self-paired (i.e., located in either a loop or bulge) to mitigate competitive inhibition with the DNAzyme. Commercially available Lipofectamine™ 3000 was used to deliver the DNAzyme constructs into classically activated macrophages, and RT-qPCR revealed the first construct had the most significant decrease in irf5 expression (P<0.0001) and downstream effects in TNF-α (P<0.01) relative to the control and other two constructs. As such, all subsequent experiments used the first DNAzyme construct encapsulated within the PCaP NP, and analysis was conducted 24 hours post-treatment due to time-dependent studies on PCaP NP-treated activated macrophages (FIG. 1). Western blot performed at this time-point demonstrates a significant downregulation of IRF5 (P<0.001), and the pro-inflammatory cytokine IL-1β (P<0.05) in the PCaP NP treated classically activated macrophages compared to the untreated group (FIG. 2), IRF5 was not detected in the unactivated macrophages.

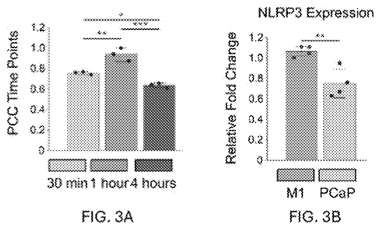

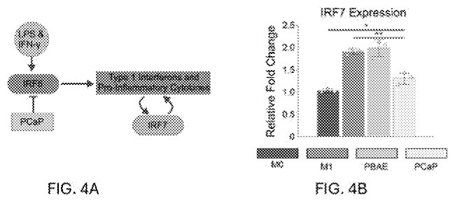

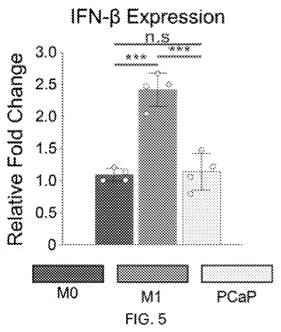

Macrophages are hard-to-transfect cells that have developed various methods to sense foreign nucleic acids. To this end, Applicant sought to verify that the PCaP NP platform could be successfully internalized by activated macrophages and effectively escape from endosomal compartments. To accomplish this goal, a dye (Alexa Fluor 647) labeled DNAzyme was delivered via the PCaP NP platform and tracked in relation to endosomes labeled with Rab5. Over a four-hour period, immunofluorescent images were analyzed to assess the distribution of DNAzymes relative to endosomal compartments. By generating a color scatter plot and calculating the Pearson correlation coefficient, reduced colocalization between the DNAzymes and endosomes were observed, proving that the DNAzymes successfully escaped the endosomal environment (FIG. 3A). Additionally, there was a significant (P<0.01) decrease in nlrp3 expression in PCaP NP-treated activated macrophages compared to the untreated condition (FIG. 3B). Previous studies have shown that the nlrp3 inflammasome marker is upregulated following the escape of cargo from late endosomal or lysosomal compartments. Conversely, lower levels of this marker are associated with early endosomal escape, suggesting that prompt release of delivered materials may help avoid activating this inflammatory pathway. The ability to promote endosomal escape from the PCaP NP platform was further demonstrated by noting a decrease in activated macrophage markers downstream of IRF5, including irf7 and ifn-β, highlighting the ability of this platform to modulate innate immune responses (FIG. 4A-B and FIG. 5).

### Example 3

### Assessing Macrophage Health and Bioenergetics via Analysis of Metabolomic and Lipidomic Markers

In this Example, metabolomic and lipidomic markers were tested to evaluate macrophage health and bioenergetics. By examining shifts in metabolic profiles and lipid composition, insight can be gain into the cellular state, energy utilization, and functional capabilities of macrophages following treatment interventions. Immunometabolism studies have revealed the methods macrophages use to generate energy vary depending on their activation state. Specifically, activation of macrophages in response to bacterial infections or viruses is metabolically demanding and relies on glucose metabolism for fast ATP production. Due to the disruption that is observed in the tricarboxylic acid (TCA) cycle, there are notable alterations in metabolites, including an accumulation of citrate and succinate that result in lipid biosynthesis, activation of the transcription factor HIF-1α, and expression of pro-inflammatory genes. Conversely, alternatively activated macrophages utilize fatty acid oxidation (FAO) and oxidative phosphorylation (OXPHOS) in addition to an undamaged TCA cycle. It has also been noted that alternatively activated macrophages have higher glycolytic capabilities than their unactivated counterpart but less so than classically activated macrophages, however, this bioenergetic pathway is not necessary to drive the anti-inflammatory state. It was hypothesized that delivering PCaP NPs to classically activated macrophages would induce a discernible shift in their bioenergetic profile. Given that IRF5 typically enhances glycolysis and stimulates the release of pro-inflammatory cytokines, downregulating this transcription factor should prompt metabolic reprogramming toward alternative energy pathways and promote an anti-inflammatory phenotype. This was accomplished by a multifaceted approach whereby quantitative NMR (qNMR) was used to quantify several water-soluble metabolites, and mass spectrometry was implemented to quantify lipids.

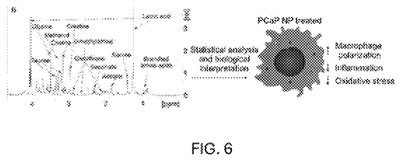

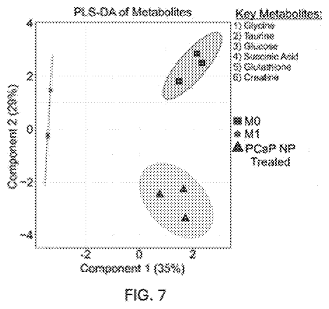

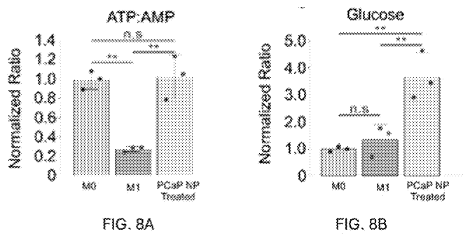

Classically activated macrophages treated with PCaP NPs were lysed, and aqueous metabolites were collected to be analyzed by qNMR (FIG. 6). From the aqueous fraction, 19 metabolites were quantified and used these metabolites to demonstrate the PCaP NP-treated macrophages manifested themselves as a distinct population that was unique form unactivated and classically activated macrophages (FIG. 7). Loading scores were determined from partial least squares discriminate analysis (PLS-DA) to identify key metabolites that aided in classifying the various macrophage populations. From the loading scores, the top 6 aqueous metabolites were identified as (i) glycine, (ii) taurine, (iii) glucose, (iv) succinic acid, (v) glutathione, and (vi) creatine (FIG. 7). Interestingly, in macrophages, glycine has been shown to promote an alternatively activated phenotype by preventing the activation of NF-κB. Similarly, taurine has been shown to prevent metabolic shifts to glycolysis while encouraging mitophagy, succinic acid promotes angiogenesis, and creatine inhibits JAK2/STAT1 pathways while upregulating STAT6 expression, all of which contribute to the anti-inflammatory phenotype. Conversely, the antioxidant glutathione has been associated with classically activated macrophages. Finally, the ATP:AMP ratio and amount of glucose per condition were quantified. It was found that the ATP:AMP ratio was significantly decreased in the classically activated conditions (P<0.01) relative to the unactivated condition. However, following PCaP NP treatment, the ATP:AMP ratio was restored to levels comparable to the unactivated macrophages (FIG. 8A). Moreover, the amount of glucose was found to be elevated significantly in the PCaP NP-treated condition relative to both the unactivated and classically activated conditions (P<0.01) (FIG. 8B).

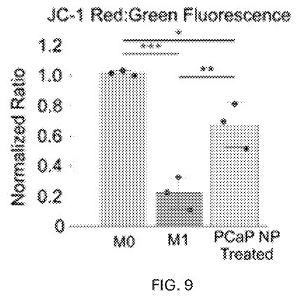

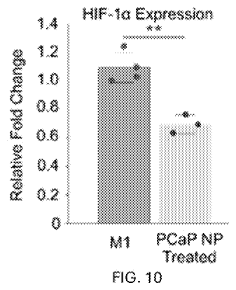

Since increased glucose uptake is associated with classically activated macrophages, the mitochondrial membrane potential was studied to gain further insight into the health and activity of this organelle in treated macrophages. Changes in mitochondrial membrane potential are linked with apoptosis, and depolarization of the membrane results in the release of apoptotic factors and a loss of OXPHOS. Quantification of fluorescent images revealed that there was a significant decrease (P<0.001) in the membrane polarization of classically activated compared to unactivated macrophages but that the membrane potential significantly increased in activated macrophages treated with PCaP NP (P<0.01) (FIG. 9). Finally, the transcription factor HIF-1α was downregulated significantly (P<0.01) in PCaP NP treated compared to classically activated macrophages (FIG. 10). Taken together, these shifts in the metabolic profile of treated classically activated macrophages are indicative of the macrophages adopting an alternatively activated phenotype, demonstrating the feasibility of PCaP NP to attenuate inflammatory responses.

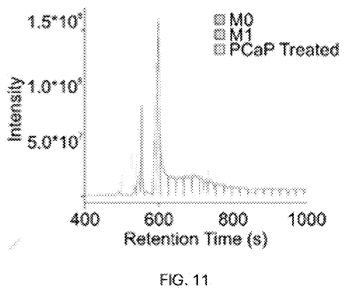

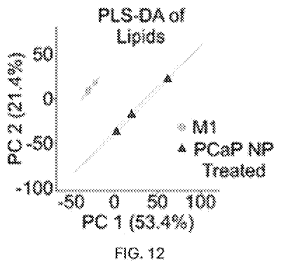

Lipidomic studies leveraging mass spectrometry were further utilized to highlight the changes in the metabolic profile of unactivated, treated, and untreated classically activated macrophages. Lipophilic metabolites were isolated from the organic phase following cell lysis and extraction prior to performing mass spec. The collected raw spectra were analyzed using MetaboAnalyst 6.0 to create and annotate a peak list that was then used to perform untargeted lipidomics analysis. Prior to performing statistical analysis, the data was filtered, normalized, transformed, and scaled (FIG. 11). PCaP NP treated macrophages were shown to possess a distinct lipidomic profile from either unactivated or classically activated macrophages (FIG. 12). After observing this unique lipidomic signature, Applicant sought to better understand which pathways were enriched in the PCaP NP treated condition.

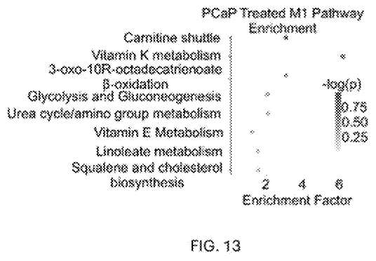

To accomplish this goal, pathway enrichment was performed using the MFN pathway library and several key pathways including: (i) carnitine shuttle (P<0.01), (ii) vitamin E (P<0.01), (iii) 3-oxo-10R octadecadienoate beta-oxidation (P<0.01), (iv) glycolysis and gluconeogenesis (P<0.01), (v) vitamin K metabolism (P<0.01), (vi) linoleate metabolism (P<0.05), and (vii) squalene and cholesterol biosynthesis (P<0.05) were identified and found to be involved in the inflammatory process (FIG. 13). The carnitine shuttle pathway is responsible for moving long-chain fatty acids, such as 3-oxo-10R octadecadienoate, into the mitochondria where they can be processed via β-oxidation to regenerate ATP at the end of the electron transport chain. Moreover, linoleic acid has been shown to activate various receptors, including PPARγ, thereby promoting an anti-inflammatory phenotype and can also be used as an energy source via FAO. While squalene is a precursor to cholesterol, this fatty acid is also implicated in the resolution of wound healing by promoting anti-inflammatory signals (i.e., IL-10, IL-13, and IL-4) and encouraging tissue remodeling while suppressing pro-inflammatory signals (i.e., TNF-α and NF-κB). Moreover, RNA-seq reveals that transcripts in the cholesterol biosynthesis pathway leading from acetyl-CoA to squalene are upregulated in the PCaP NP-treated condition while lanosterol synthase is down-regulated and should limit cholesterol synthesis. Vitamin E is one of the most effective nutrients at modulating immune response, and the metabolism of this lipid-soluble antioxidant is known to suppress pro-inflammatory signals, including NF-κB, scavenge radicals from reactive nitrogen species, and terminate lipid peroxidation. Similarly, vitamin K is known to suppress inflammatory cytokine production and NF-κB activity. Again, an increase in the glycolysis and gluconeogenesis pathways in the PCaP NP treated condition was noted, which is traditionally associated with classically activated macrophages.

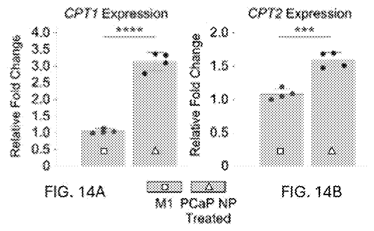

Finally, the relative amount of cpt1 and cpt2 was quantified to confirm the results observed in the pathway enrichment analysis. Both these genes are involved in the carnitine shuttle pathway as cpt1 facilitates fatty acid modification and mitochondrial entry while cpt2 promotes fatty acid metabolism via β-oxidation. These results revealed a significant increase in both cpt1 and cpt2 (P<0.0001 and P<0.001, respectively) (FIG. 14A-B) in the classically activated macrophages treated with PCaP NP. These results clearly indicate that the PCaP NP platform can guide macrophages away from a pro-inflammatory phenotype and toward an anti-inflammatory state. This is evidenced by a metabolic shift favoring fatty acid oxidation, which aligns with the functional characteristics of alternatively activated macrophages.

### Example 4

### Transcriptomic Profiles of Classically Activated and PCaP NP Treated Macrophages

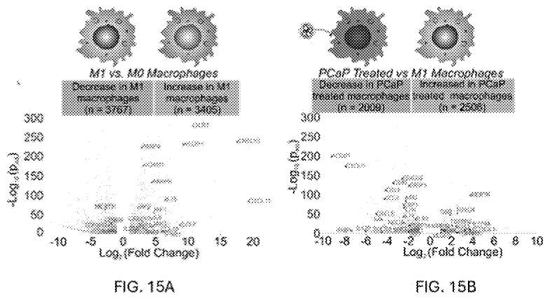

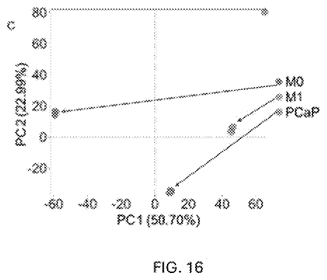

To gain insight into how the PCaP NP platform influences the transcriptomic profile of treated classically activated macrophages, the unactivated, classically activated, and PCaP NP-treated macrophages using bulk RNA sequencing were analyzed. Given the metabolomic changes that were observed in both the hydrophilic and hydrophobic metabolites, it was expected for significant changes in the phenotype of treated cells to be observed. Note that 24 hours after activation, there was an increase in pro-inflammatory chemokines, chemokine receptors interleukins, vascular cell adhesion molecules, JAK-STAT activation, and transcription factors in the classically activated compared to unactivated macrophages (FIG. 15A). Similarly, anti-inflammatory signals such as mertk, tgfßR2, and il4r were downregulated. However, when the classically activated macrophages were treated with the PCaP NP platform a decrease in many pro-inflammatory markers, including interferons, cytokines, toll-like receptors, transcription factors, and caspases and an increase in anti-inflammatory growth factors, transcription factors, interleukins, markers of alternatively activated macrophages, and tissue remodeling proteins (FIG. 15B). In fact, principal component analysis reveals the treated activated macrophages adopt a phenotype that is distinct from either the unactivated and classically activated macrophages (FIG. 16).

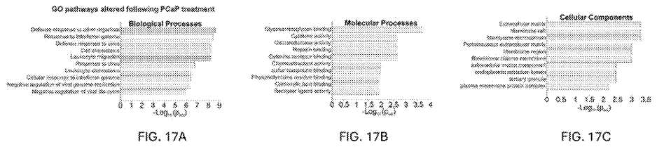

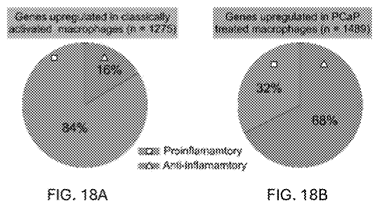

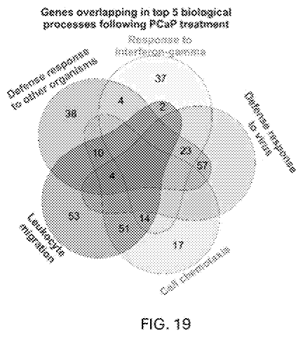

Next, the genes that controlled the major biological processes that most significantly affected following PCaP NP treatment were determined, including defense response to other organisms and response to interferon-gamma, respectively (FIG. 17A). Furthermore, following treatment with PCaP NP, the top five molecular functions increased involved recognizing, responding to, and providing chemical signals in the environment, including (i) glycosaminoglycan binding, cytokine activity, oxidoreductase activity, heparin binding, and cytokine receptor binding (FIG. 17B). Moreover, the top five cellular components affected by PCaP NP treatment in classically activated macrophages were related to extracellular signaling and include (i) extracellular matrix, (ii) membrane rafts, (iii) membrane microdomain, (iv) proteinaceous extracellular matrix, and (v) membrane region (FIG. 17C). Reactome pathway analysis also highlighted that PCaP NP-treated activated macrophages had alterations in the extracellular matrix organization, cytokine signaling, immune system, metabolism, and signal transduction. When comparing the genes that were differentially expressed between the PCaP NP-treated and classically activated conditions, there was a clear shift in the transcriptomic profile between these conditions. Of the genes involved in the inflammatory process, 84% of the differentially expressed genes (DEGs) in classically activated macrophages were pro-inflammatory, and only 16% were immunosuppressive (FIG. 18A). Conversely, in the PCaP NP-treated group, 68% of the DEGs displayed immunosuppressive characteristics, while only 32% were identified as immunoactive (FIG. 18B). Finally, the genes associated with the top five biological processes that were increased in classically activated macrophages following PCaP NP were organized in a Venn diagram and approximately 83.5% (259/310) of the genes were involved in 1 or 2 processes, and the remaining 51 were involved in 3-4 processes with only 4 genes (cxcl10, cxcl9, il6, and lyst) involved in 4 biological processes (FIG. 19).

It should be noted that the above-mentioned preferred examples are further, non-limiting, and detailed descriptions of the embodiments of the present disclosure and are only for illustrating the technical concepts and features of the present disclosure. They are provided for better understanding and implementation of the present disclosure by those skilled in the art, rather than limiting the scope of the present disclosure. All equivalent changes and modifications made according to the spirit of the present disclosure should fall within the protection scope of the present disclosure.

What is claimed is:

1. A nanoparticle comprising:
(a) a core comprising alkaline earth phosphate encapsulated oligonucleotides, and
(b) an ionizable polymer enclosing the core,
wherein the ionizable polymer is a biocompatible anionic polymer that ionizes at a pH between about 4 and about 6.5.

2. The nanoparticle of claim 1, wherein the alkaline earth phosphate is magnesium phosphate or calcium phosphate.

3. The nanoparticle of claim 1, wherein the alkaline earth phosphate core is doped with one or more divalent metal ion(s).

4. The nanoparticle of claim 3, wherein the divalent metal ion is selected from Zn²⁺ and Mn²⁺.

5. The nanoparticle of claim 1, wherein the oligonucleotide is selected from the group consisting of miRNAs, siRNAs, plasmids, shRNA, peptide nucleic acids (PNA), aptamers and DNAzymes.

6. The nanoparticle of claim 5, wherein the oligonucleotide is a DNAzyme.

7. The nanoparticle of claim 6, wherein the DNAzyme is a 10-23 DNAzyme that suppresses an mRNA encoding the protein Interferon Regulatory Factor 5 (IRF5).

8. The nanoparticle of claim 7, wherein the 10-23 DNAzyme comprises a nucleotide sequence with at least 95% sequence identity to a nucleotide sequence selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca. |

9. The nanoparticle of claim 1, wherein the ionizable polymer is a poly(amino acid)-based polymer or co-polymer, a biopolymer, a polylactide-polyethylene glycol (PLA-PEG), or a zwitterionic polymer or co-polymer,
wherein the poly(amino acid)-based polymer or co-polymer is selected from the group consisting of poly(β-amino ester) (PBAE), poly(aspartic acid-co-lysine), poly(glutamic acid-co-lysine), and poly(aspartic acid-co-histidine);
wherein the biopolymer is selected from the group consisting of gelatin type B, collagen, casein, and peptidoglycan; and
wherein the zwitterionic polymer or co-polymer is selected from the group consisting of poly(carboxybetaine methacrylate) and poly(acrylic acid-co-dimethylaminoethyl methacrylate).

10. The nanoparticle of claim 9, wherein the ionizable polymer is a poly(β-amino ester) (PBAE).

11. The nanoparticle of claim 1, wherein the nanoparticles have an average size of about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, and any range between any two of the aforementioned values, as characterized by transmission electron microscopy.

12. A method of making the nanoparticle of claim 6 comprising:
(a) mixing two microemulsions, wherein the first microemulsion comprises alkaline earth halide and a DNAzyme, and wherein the second microemulsion comprises disodium phosphate, and
(b) stirring the mixture of step (a) until alkaline earth phosphate particles encapsulating the DNAzyme are formed,
(c) incubating a solution of an insoluble polymer and a base to form a solution of activated polymer, and
(d) adding the alkaline earth phosphate particle formed in step (b) to the activated polymer solution in step (c) to form the polymer-coated nanoparticles.

13. The method of claim 12, wherein the alkaline earth phosphate encapsulated DNAzyme particles formed in step (b) are washed with a solvent comprising ethanol.

14. The method of claim 12, wherein the base is sodium acetate.

15. The method of claim 12, wherein the insoluble polymer is a poly(amino acid)-based polymer or co-polymer, a biopolymer, a polylactide-polyethylene glycol (PLA-PEG), or a zwitterionic polymer or co-polymer,
wherein the poly(amino acid)-based polymer or co-polymer is selected from the group consisting of poly(β-amino ester) (PBAE), poly(aspartic acid-co-lysine), poly(glutamic acid-co-lysine), and poly(aspartic acid-co-histidine);
wherein the biopolymer is selected from the group consisting of gelatin type B, collagen, casein, and peptidoglycan; and
wherein the zwitterionic polymer or co-polymer is selected from the group consisting of poly(carboxybetaine methacrylate) and poly(acrylic acid-co-dimethylaminoethyl methacrylate).

16. The method of claim 15, wherein the insoluble polymer is a poly(β-amino ester) (PBAE).

17. A 10-23 DNAzyme comprising a nucleotide sequence with at least 95% sequence identity to a nucleotide sequence selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca |

18. The 10-23 DNAzyme of claim 17, wherein the nucleotide sequence is selected from the group consisting of:

| (SEQ ID NO: 1) | |
|---|---|
| | gctgctaggctagctacaacgatgtctca; |
| (SEQ ID NO: 2) | |
| | aagcgcaggctagctacaacgattgctcc; |
| (SEQ ID NO: 3) | |
| | tttttaaggctagctacaacgaattcatt; |
| (SEQ ID NO: 4) | |
| | gctgctaggccagctacaacgatgtctca; |
| | and |
| (SEQ ID NO: 5) | |
| | gagctgctaggctagctacaacgatgtctcaca |


SEQUENCE LISTING (5 sequences)

SEQ ID NO: 1 - DNA, 29 bp, synthetic construct
  source 1..29: mol_type = other DNA
gctgctaggctagctacaacgatgtctca

SEQ ID NO: 2 - DNA, 29 bp, synthetic construct
  source 1..29: mol_type = other DNA
aagcgcaggctagctacaacgattgctcc

SEQ ID NO: 3 - DNA, 29 bp, synthetic construct
  source 1..29: mol_type = other DNA
tttttaaggctagctacaacgaattcatt

SEQ ID NO: 4 - DNA, 29 bp, synthetic construct
  source 1..29: mol_type = other DNA
gctgctaggccagctacaacgatgtctca

SEQ ID NO: 5 - DNA, 33 bp, synthetic construct
  source 1..33: mol_type = other DNA
gagctgctaggctagctacaacgatgtctcaca